(19) United States
(12) Patent Application Publication
(10) Pub. No.: US 2014/0076022 A1
(43) Pub. Date: Mar. 20, 2014

(54) DEVICE, A SYSTEM AND A METHOD FOR ALCOHOL MEASUREMENT

(71) Applicant: AB Halmstad Kylteknik, Halmstad (SE)

(72) Inventors: Henrik Ohlsson, Halmstad (SE); Peter Ohlsson, Laholm (SE); Hans-Erik Eldemark, Eldsberga (SE); Bengt Daniel Felix Petersson, Halmstad (SE)

(73) Assignee: AB Halmstad Kylteknik, Halmstad (SE)

(21) Appl. No.: 14/086,317

(22) Filed: Nov. 21, 2013

Related U.S. Application Data
(63) Continuation of application No. 14/115,182.

(30) Foreign Application Priority Data
Oct. 6, 2011 (SE) 1150926-2
Mar. 15, 2012 (SE) 1250247-2

(51) Int. Cl. G01N 33/497

(52) U.S. Cl. CPC G01N 33/4972
USPC 73/23.3

(57) ABSTRACT

An alcohol measuring device comprises an intake for receiving a user exhalation and a gas sensor. A connector of the measuring device is configured to be connected to a sound port of a terminal such as a cell phone, smart phone, tablet or a similar mobile device. The measuring device is arranged to transfer measurement data via said connector to said sound port of the terminal in the form of analogue signals representing sound. Duplex communication over the sound port is possible for transferring commands to the measuring device from the terminal.

### CROSS-REFERENCE(S) TO RELATED APPLICATION(S)

This application is a continuation of U.S. application Ser. No. 14/115,182, filed Nov. 1, 2013, which is the National Stage of International Patent Application PCT/SE2012/051039, filed Sep. 28, 2012, which claims priority to Swedish Application 1250247-2, filed Mar. 15, 2012 and Swedish Application 1150926-2, filed Oct. 6, 2011. Each application is incorporated herein by reference in its entirety.

### TECHNICAL FIELD

The present invention generally relates to a device, a system and a method for alcoholic measurement. More specifically, the invention relates to a alcohol measurement concept involving a measuring device for connection to a terminal, especially a mobile phone, a smart phone, a tablet or a similar mobile device.

### BACKGROUND

It is often desirable to analyze the breath exhaled by a person to test whether the breath contains certain substances. The most common example of this is to test the content of alcohol (ethanol) in a subject's breath, to determine whether the subject is likely to have consumed too much alcohol to perform certain tasks such as driving a car or operating machinery.

Known alcohol meters suffers from the drawback that they are traditionally rather large sized and expensive. Some efforts have been made to solve such drawbacks. The prior art includes some proposed solutions involving the use of mobile phones in connection with alcohol measurement, for instance US 2004081582A, KR20060124885A and KR20020070401A. However, these prior-art suggestions either require substantial modifications of the mobile phone and/or require brand specific solutions with respect to the measuring device.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

According to a first aspect of the inventive concept, there is provided an alcohol measuring device, comprising an intake for receiving a user exhalation, a gas sensor and a connector for connecting the measuring device to a terminal, such as a mobile phone, for presenting measurement data on a display of said terminal. The connector of the device is configured to be connected to a sound port of the terminal and the measuring device is arranged to transfer measurement data via said connector to said sound port of the terminal in the form of an analogue signal representing sound.

The measuring device is preferably a handheld device and the terminal is preferably a mobile terminal, especially a cell phone, a smart phone, a tablet or the like. Mobile terminals, such as cell phones are part of a daily accessory, carried by the user almost all the time. Moreover, mobile terminals allow access to data and information useful for the user. The inventive concept makes it possible to use the mobile terminals as portable data analyzing terminals. However, a problem related to mobile terminals from different manufacturers, or even from the same manufacturer, is the different types and standards of data ports and communication protocols. Thus, when attaching external devices to mobile terminals of different brands or configurations, there is traditionally a need for adapting the physical connection, the interface and the communication protocol. In other words, devices attachable to such mobile terminals must traditionally be adopted to each specific terminal, implying in costs and verities.

The inventive concept makes it possible to provide an alcohol measuring device which can be attached to a terminal for providing measurement data information without the need of using a brand-specific data port of the terminal for the connection and the data transfer. Thereby, the inventive concept allows easy adoption to several types of terminals, especially cell phones, smart phones, tablets and other mobile devices. The inventive concept makes use of the fact that almost all cell phones, smart phones, tablets and many other terminals are provided with a common standardized headset/microphone port or socket, normally a headset/microphone socket (audio port). According to the inventive concept, the measurement data is transferred from the measuring device to the terminal as one or more analogue signals corresponding to sound signals. These analogue signals are received by the sound or audio port of the terminal, and thereafter the analogue signals may be processed in the terminal in any suitable way by use of circuitry already present in the terminal for handling conventional sound signals. The result may be display to the user on the display often present on such terminals.

In a typical embodiment, the measuring device may be arranged to convert a digital signal corresponding to a measured value to an analogue signal for output through the connector of the measuring device (in the following referred to as the “sound connector of the device”). Especially, the analogue signal may comprise information transmitted through frequency changes/modulation of a carrier wave. Thus, the data may be transferred by signals corresponding to sound signals. In one embodiment, measurement data may be transferred from the measuring device to the terminal by using e.g. a low frequency or tone representing 0 and a higher frequency or tone representing 1.

The term sound should be interpreted not being restricted to any specific frequency band, i.e. including also ultra sound and infra sound.

In a preferred embodiment, the sound connector of the measuring device comprises a standard-type sound (audio) plug or sound socket, preferably a TRRS connector. A TRRS connector presents four contacts or poles: left speaker, right speaker, microphone and common ground. In such an embodiment, the microphone contact may be used for the data transfer to the terminal. The reason why it is preferable to use a 4-pole TRRS connector and not the simpler 3-pole TRS connector is the following: Today's smart phones are designed to distinguish headsets with or without microphones. This is performed by the smart phone by connecting the microphone connector to ground. Therefore, if a TRS connector would be used in the inventive measuring device, there might be situations where a smart phone would not be able to detect the connector properly and no duplex communication would be possible as described below.

In addition to the transfer of measurement data from the measuring device to the terminal, the inventive concept also allows for a two-way communication (duplex) between the measuring device and the terminal via said sound connector of the device and said sound port of the terminal.

Especially, a contact (connector portion) of the sound connector of the measuring device corresponding to the “microphone contact” of a standardized sound port of the terminal may be used for transmitting data to the terminal in the form of analogue “sound signals”, whereas the sound or speaker contact(s) of the terminal's sound port may be used for transmitting analogue “sound signals” in the opposite direction from the terminal to the measuring device. Different frequencies may be used for different purposes. In one embodiment, such two-way communication is half-duplex (one direction at the time).

In a preferred embodiment using such duplex communication over the sound port arrangement, the terminal may act as a master and the measuring device may act as a slave. The master/slave arrangement would normally have the consequence that the duplex will be half duplex.

The measuring device may further comprise at least one memory unit for storing measurement data for subsequent transfer to the terminal. The device may be arranged to store only one measurement sample (i.e. the result from one exhalation of the user) or preferably a plurality of measurement samples from a user. According to this embodiment, the measuring device may be arranged initially, in a state not connected to the terminal, to receive one or more exhalations of a user and to store the relevant measurement data in the device. The device is then subsequently connected to the sound port of the terminal in order to transfer said stored measurement data to the terminal. After completion of the data transfer, the data may be erased from the memory unit preferably automatically without user intervention.

The memory unit may in a practical embodiment be part of a processor chip in the measuring unit, such as a RAM memory. The processor chip may include a RAM memory for this use (work memory) and also a flash memory for the software controlling the processor. In other embodiments, separate memory units may be used.

The measuring device may further include means for providing a “time stamp” to each measurement sample or data, i.e. information corresponding to when the sample was taken. This embodiment is especially preferred in combination with using the above-mentioned memory unit. Thereby, a user may easily perform—by initially using only the separate handheld measuring device unconnected to the terminal—a plurality of sequentially exhalation measurements or samples at different points in time (e.g. every one hour during an evening). Thereafter, at a convenient subsequent time, the user may connect the measuring device (with the measurement data and associated time stamp data stored therein) to the terminal (e.g. a cell phone) in order to transfer all the measurement data including the time stamp information to the terminal.

It may be possible to include the time stamp aspect also in a simpler embodiment where each measurement is transferred directly to the terminal without any data storage.

The connector of the measuring device may be one of a TRS (tip, ring, sleeve) connector, TRRS (tip, ring, ring, sleeve), an audio plug, phone plug, stereo plug, mini-plug, or a mini-stereo audio connector.

In one embodiment, the measuring device may comprise a rechargeable power source. Normally, the battery will not be charged by the terminal via the sound port.

In a preferred embodiment, the measuring device further comprises a processor unit controlling the operation of the device. The processor may control the measurement performed by the sensor, control various internal data transfer operations and storage in the device, and control the communication with the terminal. Also, the processor may control the interaction with the user by light and/or sound signalling arrangements. The processor may also perform some initial measurement of the alcohol concentration level. Further analysis of the measurement data may be performed in the terminal after the data transfer.

According to a second aspect of the inventive concept there is provided an alcohol measuring system, comprising an alcohol measuring device as described above and a terminal provided with a sound port for receiving measurement data from the measuring device in the form of analogue sound-representing signals.

The terminal may be a mobile terminal, such as a cell phone, a smart phone, a tablet or the like, provided with a display for presenting results to the user based on measurement data received from the measuring device and as suitably processed in the terminal before presentation. Preferably, the mobile terminal contains an application for controlling the communication with the measuring device and for controlling the processing and evaluation of the received measurement data and the display of user information on a display of the terminal.

The terminal may be provided with means for establishing the position of the terminal (GPS) in order for the terminal to establish current alcohol rules or limits in the relevant area/country. Such rules may be presented to the user in combination with the measurement data and may also be used to estimate when the alcohol level has decreased to a sufficient low level corresponding to said rules.

According to a third aspect of the inventive concept, there is provided a method for use in alcohol measurement, said method comprising:
providing a measuring device having a gas sensor;
performing an alcohol measurement using said device;
transferring corresponding measurement data in the form of analogue signals to a terminal via a sound port of said terminal; and
presenting information on a display of said terminal, based on measurement data received.

In a preferred embodiment of the method, the initial measurement is performed when the device is not connected to the terminal. The measurement data is temporarily stored in the measuring device. Subsequently, at a point of time convenient to the user, the measuring device is connected to the sound port of the terminal and the stored measurement data is transferred as analogue “sound signals” to the terminal. Further evaluation of the measurement data may take place in the terminal.

When using a memory for storing the measurement data, the user may perform two or more alcohol measurements at different points in time before connecting the device to the terminal. The method may then further comprise storing data associated with the points in time (time stamps) when the respective measurements are taken and transferring corresponding time stamp information together with the measurement data to the terminal.

### DESCRIPTION OF THE DRAWINGS

The inventive concept, some non-limiting embodiments and further advantages of the inventive concept will now be further described with reference to the drawings.

FIG. 1 illustrates an exemplary alcohol measuring system according to one embodiment of the inventive concept;

FIGS. 2a and 2b schematically illustrate embodiments of a measuring device according to the inventive concept;

FIG. 3 is an exemplary block diagram over some electronics of the measuring device;

FIG. 4 illustrates a sound connector used in the devices in FIGS. 2a and 2b;

FIG. 5 is an exemplary block diagram over the electronics of the terminal;

FIG. 6 is a front view of screen of a terminal, and

FIG. 7 illustrates methods steps according to one embodiment of the invention.

FIGS. 8a-8d illustrates examples of the application user display/interface.

### DETAILED DESCRIPTION

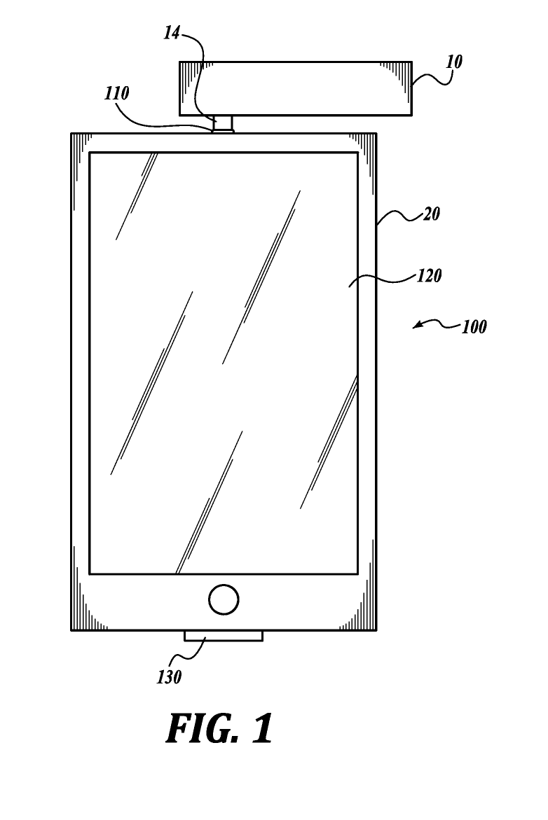

FIG. 1 schematically illustrates an embodiment of a measuring device 10 attached to a terminal 20, together forming a system 100. The device 10 is configured as a separate unit, attachable to and detachable from the terminal 20. Preferably, the size of the measuring device allows it to be used as a handheld device. As an non limiting example, the dimensions of the measuring device could be similar to the dimensions of a USB stick. The terminal 20 is in this embodiment depicted as a cell phone (smart phone) with an earphone/microphone socket 110, a display 120, and a data port 130. In general, the terminal to be used in the inventive concept will be a terminal having a standardized sound port 110.

In the example of FIG. 1, the mobile terminal 20 to which the portable measuring device 10 is connected can be but is not limited to, a cell phone, such as Apple's iPhone, Android or a suitable OS based cell phone, any other portable electronic devices or tablets, such as Apple's iPod Touch, Apple's iPad, and mobile devices based on Google's Android operating system, and any other portable electronic device, such as personal computers, that includes software, firmware, hardware, or a combination thereof that is capable of at least receiving the signal, decoding if needed, exchanging information with information server to provide relevant information.

Typical components of mobile terminal 20 may include but are not limited to a processor, persistent memories like flash ROM, random access memory like SRAM, a camera, a battery, LCD driver, a display, a cellular antenna, a speaker, a Bluetooth circuit, and WIFI circuitry, where the persistent memory may contain programs, applications, and/or an operating system for the mobile device.

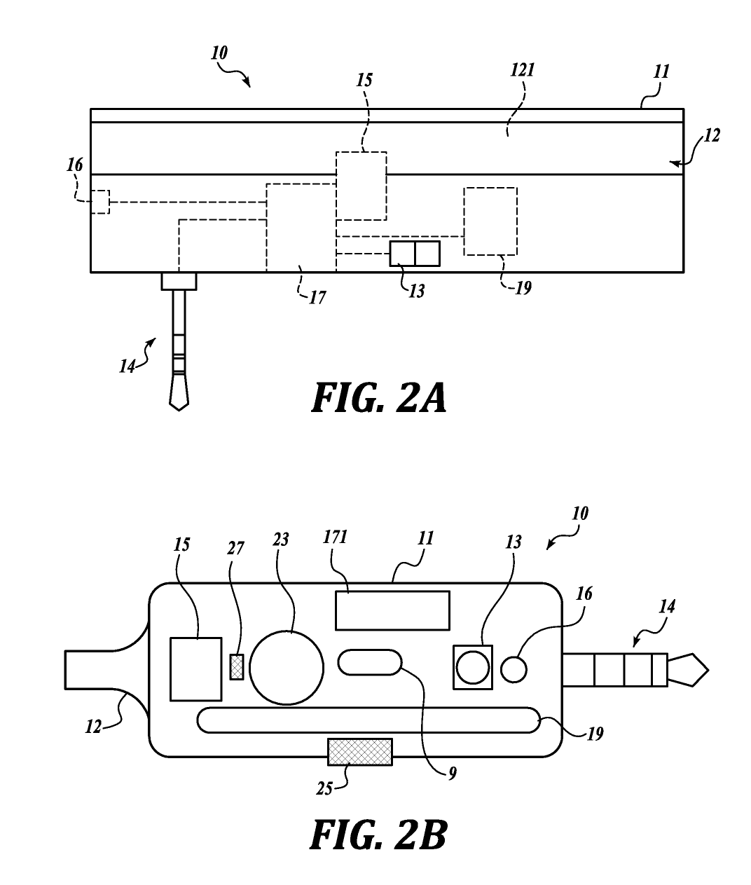

FIGS. 2a and 2b schematically illustrate two alternative designs of the measuring device 10, shown in a condition unconnected to the terminal 20. The embodiments mainly differ in the position and direction of the plug 14.

As shown in FIGS. 2a and 2b, the measuring device 10 comprises a signal plug 14 in the form of a conventional 4-pole TRRS audio plug. In the following, the signal plug 14 will be referred to as “the sound connector 14” of the device 10. The sound connector 14 is used at least for transferring data from the measuring device 10 to the terminal 20. In the preferred embodiment, the sound connector 14 is also used for transferring data in the opposite direction, e.g. command signals and other optional signals from the terminal to the device. In addition, the sound connector 14 may be used as shown in FIG. 1 for physically attaching and supporting the measuring device 10 when connected to the terminal 20 such that no other physical connection or support is needed.

According to the inventive concept, the measuring device 10 functions as an alcohol meter (alco-meter). An alco-meter, as the term used herein, refers to a device for breath-analyze, which is a device for estimating blood alcohol (ethanol) content (BAC) from a breath sample.

A handheld measuring device 10 is shown to comprise at least a housing 11, having an exhalation intake 12, a switch or function button 13 for activating/deactivating the device 10 (e.g. from a sleep mode) and for initiating a measurement mode, a signal plug 14 extending from the housing 11, and an optional signalling arrangement 16. In the embodiment in FIG. 2a, a pipe 121 is arranged inside the housing 11 extending from the intake 12 and used for exhaling into the pipe 121.

A gas sensor 15 is arranged in such a way that the blown air is displaced on or by it. The gas sensor 15 is preferably of low-power consumption type considering the limited power available in the device 10, especially when used as a separate handheld device during the measuring operation. In some embodiments, internal structure may include a wall or the like against which the blown air strikes during use, where after the blown air flows around said wall and thereafter reaches the sensor. Such an embodiment may be preferred since the blown air thereby does not hit the sensor directly and the measurement date obtained may be more reliable.

The device 10 further comprises electronics 17 and a power source 19 in the form of a rechargeable battery. The power source 19 is used to power the electronics, the sensor and the signalling arrangement. In some embodiments a charger socket 25 (FIG. 2b), e.g. a mini USB connector) may be provided to charge the rechargeable power source 19.

The signalling arrangement 16, which may comprises a light emitting diode (LED) may be used to indicate that the device is on, different states or error indication, e.g. using illumination with different colours. The LED may indicate device activation status, battery charge status, user exhalation prompt or user stop-exhalation prompt. The signalling may also include an audio signalling unit (sound generating unit) 23.

The measuring device 10 may be is configured to generate an alarm (visual, audible) based on the comparison of the measured value and a limit value stored in the memory. The limit value may be provided through the plug from the terminal when attached. FIG. 2b illustrates a sound unit 23 for this purpose.

Reference numeral 27 indicates a crystal based clock used for associating each exhalation sample with a real-time time stamp. This operation will be further described below. Further, a temperature sensor 29 is arranged for adjusting the measurement if the environmental temperature is to high or low.

In FIGS. 2a and 2b, the housing 11 of the device 10 is designed to extend in one direction from the signal plug or sound connector 14. In some embodiments, sound connector 14 may be retractable (e.g. slidable or foldable) into the housing 11. In some embodiments, the signal plug 14 is configured to extend beyond housing 11 in order to accommodate connection with mobile terminals 20 (e.g. cell phones) having cases or having a recessed plug-in socket, wherein the socket can be but is not limited to a microphone input socket or a line in audio input of the terminal.

It should especially be noted that the sound connector 14 of the measuring device 10 may be have a different configuration than the illustrated 4-contact TRRS type plug. As an alternative, the sound connector 14 may be a female TRS-type or TRRS-type socket connectable to the terminal via a separate cable. However, for today's smart phones, the TRRS type plug is preferred since present smart phones are designed to “listen” on the microphone pole or contact of the plug. It may also be possible to use different kinds of adapters between the device 10 and the terminal 20, i.e. to connect the measuring device 10 indirectly to the terminal 20.

The housing 11 of device 10 can be made of a non-conductive material such as plastic so that the device 10 will not interfere with the operation of the terminal 20. This will apply especially if the device 10 is in a terminal-connected state during the actual measurement. Such choice of material is important since the outer case of certain mobile terminals is conductive and serves as an antenna the operation of which may potentially be interfered with if a metal case of the device gets in touch with the housing of a terminal.

According to the inventive concept, the device 10 is be used for breath-analyse in the form of an alco-meter. An exhalation type alco-meter does not directly measure blood alcohol content or concentration, which requires the analysis of a blood sample. Instead, it estimates BAC indirectly by measuring the amount of alcohol in user's breath. Different technologies may be used for this purpose: infrared spectrophotometer technology, electrochemical fuel cell technology, etc. or a combination of the two. Hand-held field testing devices are generally based on electrochemical platinum fuel cell analysis. Thus, the sensor may be one of infrared spectrophotometer, electrochemical fuel cell, a combination thereof or any other suitable sensor for the purpose.

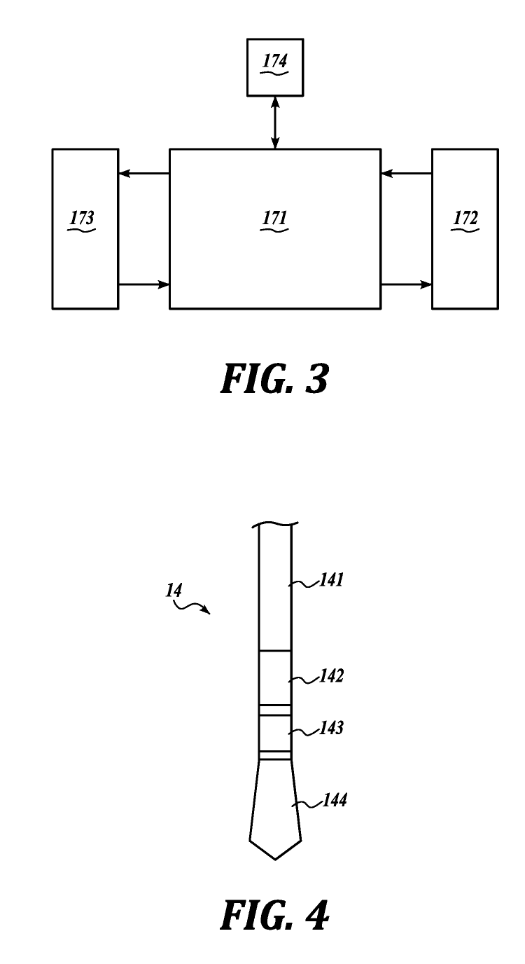

FIG. 3 illustrates schematically the electronic component portion 17 of the device 10. According to this exemplary embodiment, the electronics comprises a controller 171, an input unit 172, an output unit 173 and a memory unit 174. The controller 171, implemented as microprocessor, controls the various functions of the measuring device. It controls all sound signal communication with the terminal, it performs the measurements using the sensor 15, it controls the communication with the user (light and sound) and it detect user actions such as user operation of button 13.

The input unit 172 receives signals correspond to the sensed gas levels from the sensor 15 and provides them to the controller 171. The output unit 173 receives signals from the controller 171 corresponding to the measured levels and converts them to the sound signals (described below) and outputs them through the sound connector 14. The output unit 173 may also control the LED 16. The memory unit 174 may store instructions for operations of the controller and data for comparison. The memory unit 174, or an additional memory unit, may store sample and time stamp data. Obviously, controller, input and output units and the memory may be realized as one single circuit.

Although the diagrams depict components as functionally separate, such depiction is merely for illustrative purposes. It will be apparent that the components portrayed in this figure can be arbitrarily combined or divided into separate software, firmware and/or hardware components. As described above, the microprocessor chip used in the device may include a flash memory for storing the software and a RAM memory used as a work memory for storing the measurement data and time stamp data.

The operation of the output unit 173 of measuring device 10 is as follows: digital signals corresponding to measured values from the sensor 15 received from the controller 171 are converted into sound representing signals, i.e. analogue signals, e.g. using Frequency-shift keying (FSK). FSK is a frequency modulation scheme in which digital information is transmitted through discrete frequency changes of a carrier wave. Other modulation techniques may be used.

The output unit 173 may also receive sound signals from the terminal's 20 through the sound connector 14 and convert them into digital signals. These incoming signals may comprise commands and instructions to the measuring device 10 from the terminal 20. Thus, the digital signals are converted to “tones” with different frequencies representing a digital level (0 and 1). The measuring device 10 transmits data to the terminal via the terminal's sound port (microphone contact) through the sound connector 14. When using duplex, the measuring device 10 receives data via the earphone contacts (left and/or right). Normally, half duplex will be used.

The generated sound signal is provided through the connector 14 to the terminal 20. Here, the sound connector 14 can be but is not limited to a TRRS (tip, ring, ring, sleeve) connector also known as an audio plug or jack, phone plug, stereo plug, mini-plug, or a mini-stereo audio connector. The sound connector 14 may be formed of different sizes such as miniaturized versions that are 3.5 mm or 2.5 mm. An exemplary embodiment of the connector 14 is illustrated in FIG. 4. The connector 14 comprises four conductive parts or contacts: common ground 141, microphone 142, left speaker/sound 143 and right speaker/sound 144. This means that the measuring device 10 may transmit using the microphone contact and receive using one of or both the left and right speaker contacts.

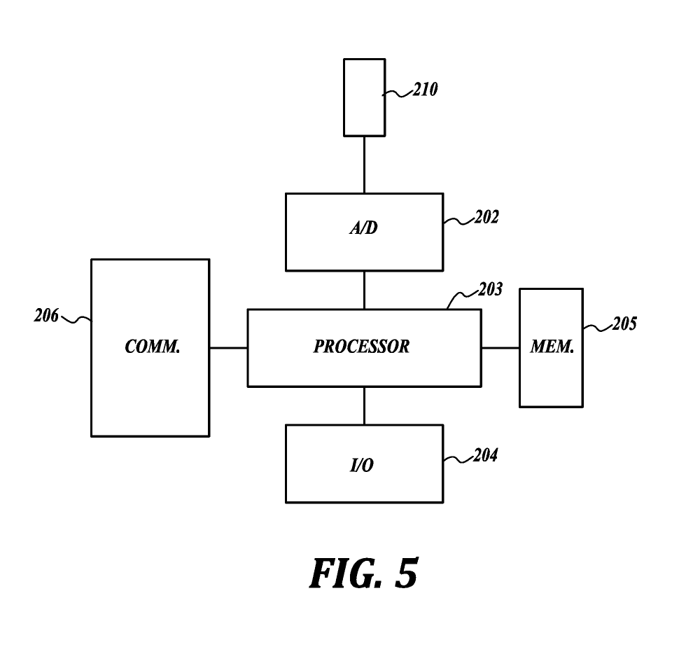

FIG. 5 schematically illustrates the electronics of terminal 20. The electronics may comprise a processor 203, an A/D converter 202, an I/O interface 204 and memory unit 205. The various parts may of course be integrated in one controller circuit. In case the terminal is a communication terminal, it may comprise a communication unit 206. The function of communication unit is assumed to be well known to a skilled person and not detailed herein. The A/D converter 202 is shown connected to a sound port 110 of the terminal 20.

The analogue “sound” signal is received from the measuring device 10 at sound port 110 and the A/D converter 202 which converts the analogue signal to a digital signal to the processor 203. The processor 203 may process the signal to determine the level of gas. The level may be compared to a value stored in the memory 205 or received from a database using communication unit. The I/O unit 204 may be configured to control input-output to various interfaces of the terminal. For example, the display, control keys and other parts interacting with a user may be controlled by the I/O unit 204 with respect to the signals received from the processor 203.

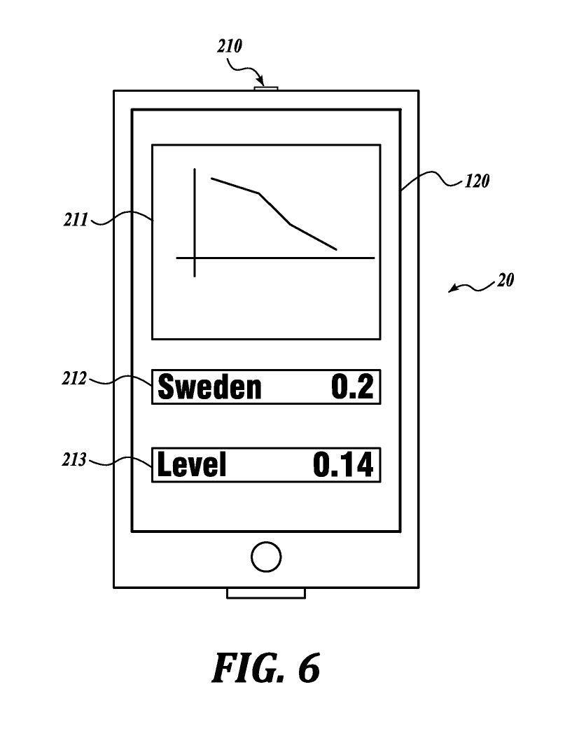

FIG. 6 illustrates, in a schematic way, an exemplary terminal 20 in the form of a smart phone, comprising a display 120 for providing information to a user. According to this example, the terminal 20 is arranged to display level of alcohol measured by the measuring unit 10. The display may comprise different fields for different types of information input and output. According to the example, a first field 211 displays a number of measured values as a graph (time-level). A second field 212 is to receive input about the region and/or automatically display the region with respect to detection using GPS and/or network positioning. This function may be helpful if the user is travelling and does not have information about the alcohol level of the present place. The information may be brought automatically from a service provider or stored in the memory. A third field 213 is used to display the current level, and may use colours for additional information, e.g. red for alarm, green for acceptable level etc. The terminal may also generate sound alerts or information.

Thus, the terminal may be pre-programmed or be provided with an application (“app”) to execute the displaying of the measurement results. The app may be downloaded from a service provider such as AppStore of Apple Inc. or Android Market or provided in the terminal, for executing the retrieval of measured values and displaying relevant information. In one embodiment, the application may run in an idle mode and start when the measuring device is attached to the terminal.

The measuring device (10) may contain a memory containing brand information, such as a company name and/or logotype, in order to transfer a corresponding brand information to the terminal (20) for display on the terminal when the device is connected thereto.

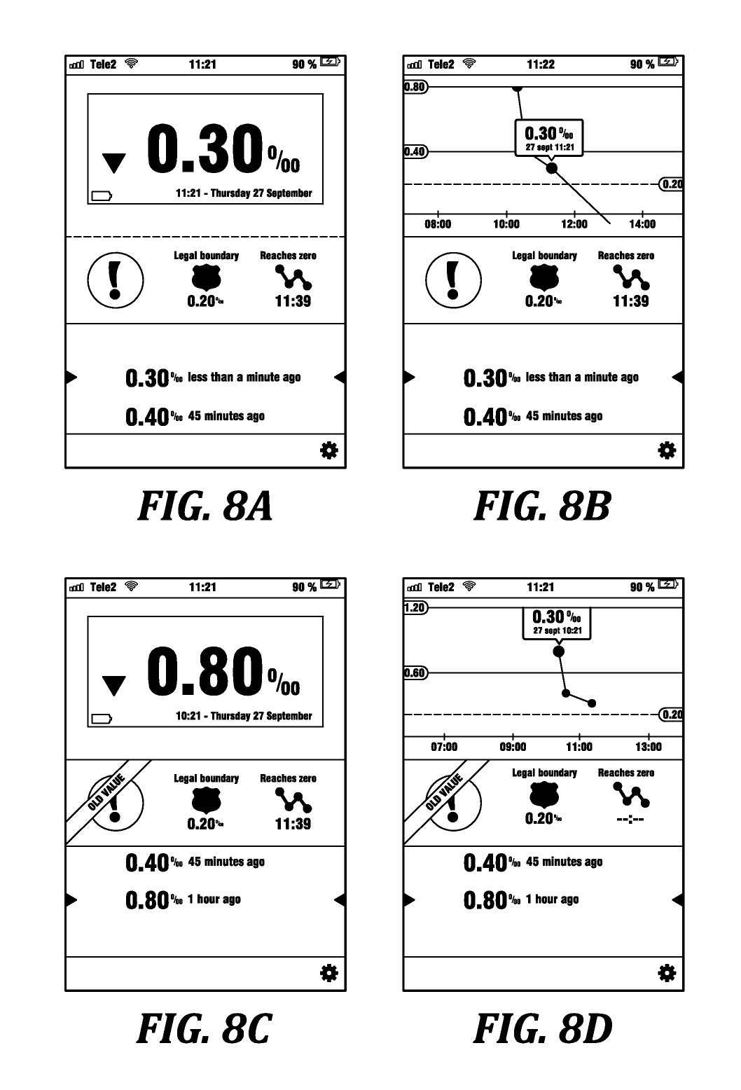

FIGS. 8a-8d illustrate examples of user interface on the display of the terminal 20, in this case a smart phone. FIG. 3a illustrates the most recent measurement value (0,30). The exclamation mark shows that the value is “illegal” (>0,2). The user also receives an estimated time when the level reaches zero. FIG. 8b shows the corresponding diagram. In the upper part of the screen, a diagram (level vs. time) illustrates the measured alcoholic level at different points in time. The horizontal dashed line (0,20) represents the legal limit for driving in the region/country as identified by the cell phone's GPS function. The lower part of the display shows details about the different measurements (level and time).

FIGS. 8c and 8d correspond to FIGS. 8a and 8b respectively, but for an older measurement data (0,8). The text over the exclamation mark shows that this is an old value not to be used.

The measuring device 10 may be provided with a unique identity to attach it to a specific user and/or application/application site, e.g. a vehicle, work site, region, etc. The identity may then be provided to the terminal and measured values for a certain user processed (stored).

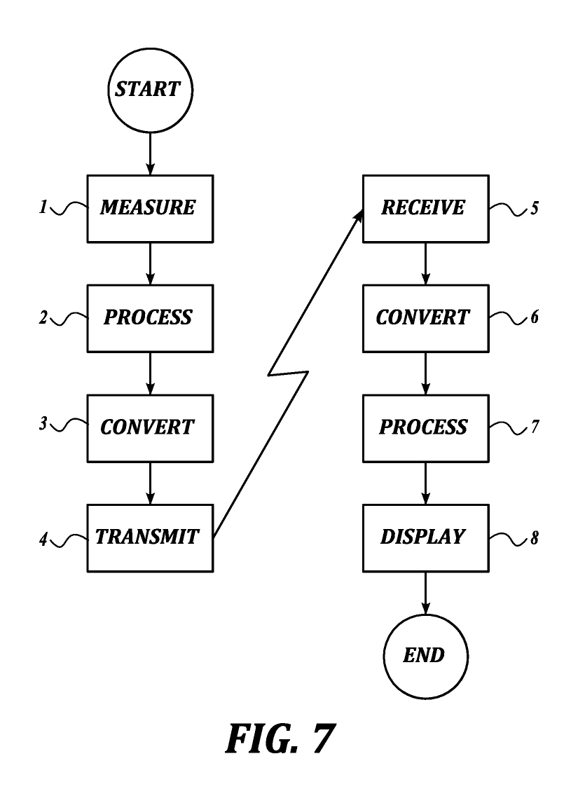

FIG. 7 illustrates some exemplary step embodiments of the invention. In a first step (1) a alcohol measurement is performed, the result is processed (2) in the controller and then converted (3) from digital form to analogue signal, comprising frequency changes of a carrier wave in the measuring device and transmitted (4) through the plugs to the terminal. The terminal receives (5) the analogue signal and converts (6) it to a digital signal and processes (7) the signal and the result is displayed (8).

The measuring device may present the following modes of operation:

Sleep Mode

The device 10 will first be in its sleep mode (standby). In this mode it consumes less power. It performs no functions but keeping track of time.

Normal Operation Mode

When the user activates the function button 13, the device 10 will be activated from its sleep mode. It will make a pip sound and the LED 16 will be giving a green flash. The device 10 is now ready to communicate with the terminal 20 if the device 10 is connected via the connector 14.

If the user instead decides to perform a measurement, he pushes button 13 again for about 2 seconds. The device will then switch to its next mode “pre-heating”.

If the user does not perform any action or does not connect the device 10 to the terminal, the device 10 will automatically return to its sleep mode.

Pre Heating Mode

In this mode, the processor 171 will control the voltage level used for the sensor preheating. During the actual preheating, the voltage is extra high in order to more quickly get the heat up. After a short period (5-10 seconds), the unit switches to its next mode of operation—normal heating. During the preheating period, the LED 16 is red.

Normal Heating Mode

In this mode, the processor 171 will control the voltage level for the sensor 15 to a level used during the actual measurement. Once the output from sensor 15 has stabilized (i.e. the alcohol concentration signal), the device is ready for user exhalation. The LED 16 is now green. When the user starts the exhalation, the device switches to its measuring mode.

Measuring Mode

During the measuring mode (i.e. during the user exhalation phase), the microprocessor will “record” the alcohol concentration signal and it will store the actual “curve” locally in the processor memory.

During the exhalation, the device 10 will generate a sound (23) and flash red (16) during about 5 seconds. Thereafter, the measurement is finalized and it is time for the processor 171 to perform “calculations”.

In one embodiment, the measuring device may be arranged to count the number of measurements. Based on the counted number, the user may at an appropriate count level decide to initiate a calibration of the sensor, e.g. by sending the measuring device to a manufacturer for calibration.

Calculation Mode

The processor retrieves relevant information from the measurement curve related to the alcohol concentration level. This measurement data is packed into a measurement package (measurement data). At a later time, the terminal 20 will read the contents of this data package.

This data package will also include a time stamp from the processor 171 using the clock 27. The measurement package is stored in the memory for later retrieval.

Thereafter, the processor 171 will perform a rough analysis whether the associated measurement contained alcohol or not. The LED 16 will present a red or green light depending on the result of this rough analysis. In an embodiment, the microprocessor used in the device 10 will not be very powerful, and therefore the calculations performed in the device will be limited. The device may be able to perform a rough calculation to the extent that it may indicate to the user (before further analysis by the terminal) whether the exhalation contained any alcohol at all, or as an alternative whether the exhalation contained an alcohol concentration exceeding a predetermined reference level, such as a level determined by the law in the actual region. Thereby, the user will be able to make a first determination based on the direct indication from the device. The more exact calculation—based on logarithmic calculations, may be performed in the more powerful terminal 20.

The device 10 will thereafter return to its normal operation mode.

Data Transfer Mode

The user subsequently connects the measuring device 10 to the terminal 20 using the sound connector 14 and the sound port 110 of the terminal 20. In a preferred embodiment the arrangement is such that the terminal 20 will be able to distinguish the measuring device 10 from a conventional headset. This function may be implemented by the device 10 by transmitting a “known” or predetermined identification sound signal from its sound connector 14. This identification sound signal is detected and verified by the terminal 20, which in response thereto will initiate its communication with the device 10 for receiving measurement data. A conventional headset will not generate this sound signal when connected to the terminal. Without this verification function, the communication sound signal generated by the software the application—if activated in the terminal—would be sent to a headset and create an unpleasant situation for the user.

The terminal will now receive measurement data associated with the measurement most recently performed, or associated with a plurality of recently performed exhalations depending on how many measurements the user has performed since the last connection. The arrangement may be such that the measurement data is erased from the device 10 after completed data transfer.

Calculation in Terminal

When the terminal 20 (cell phone) has received the measurement data or package(s) form the device 10, it will calculate the exact alcoholic concentration and display the result to the user on the terminal display.

The general principle for a possible communication between the device and the terminal will now be described.

Plug-In & Verification

When the device 10 is plugged into the terminal 20, the application in the terminal will detect that the device 10 is connected. The terminal 20 will now initially verify that it is actually a correct or “proprietary” measuring device being connected. The verification is performed by the terminal sending a “device ID” request. In response, the device 10 sends its ID to the terminal 20.

Data Transfer

The terminal will then send a request information on the number of measurement data stored in the device 10. The device 10 sends the relevant information about the number of measurements. Then, the terminal 20 will send a request command for the device 10 to send all measurement data (including time stamp data).

Data Package Contents

In a preferred embodiment of the communication protocol, one data package (sound transfer) comprises the following:
A synchronisation signal used for calibration of the data transfer rate.
Package length: Information to the terminal on the amount of data to expect.
The actual data package including: command (read or write); data information and a CRK check.

Master/Slave

In the preferred embodiment, the terminal (cell phone) is the master and initiates all commands. The measuring device responds to the commands received. When receiving a write command, the device confirms by sending an ACK signal so that the master knows that the device 10 has understood the command. When receiving a read command, the device 10 respond by transmitting the requested information.

Identification Signal

In order for the terminal 20 to be able to distinguish the measuring device 10 from a conventional headset/microphone, the device 10 may transmit a continuous tone detected by the application in the terminal 20. The terminal will then be able to determine that this is not a headset and it will initiate the above-described communication protocol. Without this arrangement, the user may hear the sound communication from the application in the headset.

The terminal 20 may be equipped with additional communication means, such as Bluetooth and/or Infrared (IR), and the measuring device may also comprise such communication means and communicate with the terminal using radio, IR and/or analogue communication as described above for data exchange. For instance, the data transmission may be performed over the sound port whereas the terminal commands in the opposite direction may be transmitted over a different channel, such as a Bluetooth channel.

The various embodiments of the present invention described herein is described in the general context of method steps or processes, which may be implemented in one embodiment by a computer program product, embodied in a computer-readable medium, including computer-executable instructions, such as program code, executed by computers in networked environments. A computer-readable medium may include removable and non-removable storage devices including, but not limited to, Read Only Memory (ROM), Random Access Memory (RAM), compact discs (CDs), digital versatile discs (DVD), etc. Generally, program modules may include routines, programs, objects, components, data structures, etc. that perform particular tasks or implement particular abstract data types. Computer-executable instructions, associated data structures, and program modules represent examples of program code for executing steps of the methods disclosed herein. The particular sequence of such executable instructions or associated data structures represents examples of corresponding acts for implementing the functions described in such steps or processes.

Although not part of the claimed inventive concept, the principle of using a sound port of a terminal (e.g. a cell phone, a smart phone a tablet, a laptop, or the like) for receiving data from a separate measuring device, and optionally also for duplex communication, may also be possible to use for measuring other physical quantities, such as other gases, medical measurements, temperature, radiation, illumination, etc. This more general aspect of the analogue signalling principle or protocol between a separate measuring unit and a terminal having a sound port may be the subject of a divisional application. In such implementations, the terminal may also be in another form, such as a vehicle or the like to which the device is connected. In such case, the terminal may be provided without any display.

While illustrative embodiments have been illustrated and described, it will be appreciated that various changes can be made therein without departing from the spirit and scope of the invention.

The embodiments of the invention in which an exclusive property or privilege is claimed are defined as follows:

1. An alcohol measuring device, comprising an intake for receiving a user exhalation; a gas sensor; and a connector for connecting the measuring device to a terminal, such as a mobile phone, for presenting measurement data on a display of said terminal, wherein said connector is configured to be connected to a sound port of said terminal, and wherein the device is arranged to transfer measurement data via said connector to said sound port of the terminal in the form of analogue signals representing sound.

2. A measuring device as claimed in claim 1, wherein said measuring device is arranged to both transfer measurement data to the terminal via said sound port and to receive data, such as command data, from said terminal via said sound port.

3. A measuring device as claimed in claim 2, wherein said terminal is a mobile device, such as mobile phone, a smart phone, a tablet or the like.

4. A measuring device as claimed in claim 1, further comprising a memory unit for storing measurement data for subsequent transfer to said terminal.

5. A measuring device as claimed in claim 4, wherein said measurement data, as stored in said memory unit of the measuring device and as transferred to the terminal, comprises data from two or more exhalations.

6. A measuring device as claimed in claim 4, further comprising clock means for providing time information associated with the measurement data stored in the device, said time information being transferred together with the measurement data to the terminal.

7. A measuring device as claimed in claim 4, wherein said measuring device in use is arranged to initially, in a state not connected to the terminal, receive one or more user exhalations and store relevant data in said memory unit, and arranged to subsequently be connected to the sound port of the terminal for transfer said stored measurement data to the terminal.

8. A measuring device as claimed in claim 1, further comprising a rechargeable power source.

9. A measuring device as claimed in claim 1, wherein said device is arranged to transfer information to said terminal via said connector and said sound port allowing the terminal to determine that the sound port is connected to a measuring device and not to conventional headset/speakers/microphone.

10. An alcohol measuring system, comprising an alcohol measuring device according to claim 1, and a terminal provided with a sound port for receiving measurement data from the measuring device in the form of analogue sound-representing signals.

11. A system as claimed in claim 10, wherein said terminal is a mobile terminal, such as a cell phone, a smart phone, a tablet or the like, provided with a display for presenting result to the user based on measurement data received from the measuring device.

12. A system as claimed in claim 11, wherein the terminal is configured to provide information based on the position of the terminal.

13. A method for use in alcohol measurement, said method comprising:
providing a measuring device having a gas sensor;
performing an alcohol measurement using said device;
transferring corresponding measurement data in the form of analogue signals to a terminal via a sound port of said terminal; and
presenting information on a display of said terminal, based on the measurement data received.

14. A method as claimed in claim 13, wherein step of performing an alcohol measurement using said device is performed in a state where the measuring device is not connected to the terminal, said method further comprising:
storing measurement data in the measuring device; and
connecting the measuring device to said sound port of the terminal;
wherein the step of transferring the measurement data comprises transferring said stored measurement data.

15. A method as claimed in claim 14, wherein the step of performing an alcohol measurement comprises performing two or more alcohol measurements at different points in time, said method further comprising storing data associated with the points in time when the measurements are taken and transferring corresponding time stamp information together with the measurement data to the terminal.

16. A measuring device as claimed in claim 1, wherein said terminal is a mobile device, such as mobile phone, a smart phone, a tablet or the like.

17. A measuring device as claimed in claim 2, further comprising a memory unit for storing measurement data for subsequent transfer to said terminal.

18. A measuring device as claimed in claim 3, further comprising a memory unit for storing measurement data for subsequent transfer to said terminal.
